(19) United States
(12) Patent Application Publication
(10) Pub. No.: US 2008/0091239 A1
(43) Pub. Date: Apr. 17, 2008

(54) Cardiac assist device and method using epicardially placed microphone

(75) Inventors: Anna-Karin Johansson, Vallentuna (SE); Kenth Nilsson, Akersberga (SE); Cecilia Tuvstedt, Stockholm (SE); Kjell Noren, Solna (SE); Anders Bjorling, Solna (SE); Andreas Blomqvist, Spanga (SE); Berit Larsson, Dandervd (SE); Sven-Erik Hedberg, Kungsangen (SE); Karin Jarverud, Solna (SE); Nils Holmstrom, Jarfalla (SE)

Correspondence Address: SCHIFF HARDIN, LLP;PATENT DEPARTMENT, 6600 SEARS TOWER, CHICAGO, IL, 60606-6473, US

(73) Assignee: St. Jude Medical AB

(21) Appl. No.: 11/582,211

(22) Filed: Oct. 16, 2006

(51) Int. Cl. A61N 1/36; A61N 1/362; A61N 1/39

(52) U.S. Cl. 607/4

(57) ABSTRACT

In a cardiac assist device and method, a microphone is placed in contact with the epicardium of the heart of a patient, and heart and lung sounds are simultaneously detected at the placement location of the microphone. The heart and lung sounds are automatically analyzed to set an appropriate cardiac therapy for the patient.

### BACKGROUND OF THE INVENTION

1. Field of the Invention

The present invention is related to implantable cardiac assist devices, in particular implantable devices that deliver pacing and/or cardioversion/defibrillation therapy to a heart of a patient.

2. Description of the Prior Art

It is of course well known to detect heart and lung sounds extracorporeally, using a stethoscope or a heart sound microphone that is placed on the skin of the patient's chest. Based on experience, a physician listening to these heart and lung sounds can make at least a preliminary diagnosis of the possibility of abnormal heart or lung conditions. Typical signals, which a physician can hear and differentiate, include a sound (designated herein as sound S1) that indicates the beginning of systole, which is created when the increase in ventricular pressure during contraction exceeds the pressure within the atria causing a sudden closing of the tricuspid and mitral valves. The ventricles continue to contract throughout systole, forcing blood through the aortic and pulmonary (semilunar) valves. At the end of systole, the ventricles begin to relax, the pressure within the heart becomes less than the pressure in the aorta and pulmonary artery, and a brief backflow of blood causes the semilunar valves to snap shut, producing a further detectable sound (designated herein as sound S2). An abnormally loud S1 may occur in connection with any condition associated with increased cardiac output, such as fever, exercise, hyperthyroidism, anemia, etc., as well as during tachycardia and left ventricular hypertrophy. A loud S1 is also characteristically heard with mitral stenosis, as well as when the P-R interval of the ECG is short.

An abnormally soft S1 may be heard in association with mitral regurgitation, heart failure, and first-degree AV block (prolonged P-R interval). A split S1 is frequently heard along the left lower sternal border, and generally is considered normal. A prominent, significantly split S1, however, may be associated with right bundle branch block (RBBB). Beat-to-beat variation in the loudness of S1 may occur in the case of atrial fibrillation and third degree A-V block.

An abnormally loud S2 is commonly associated with systemic and pulmonary hypertension.

A soft S2 may be heard in the later stages of aortic or pulmonary stenosis.

Reversed S2 splitting (S2 split during expiration, but a single sound during inspiration) may be heard in some cases of aortic stenosis, but also is common in the case of left bundle branch block (LBBB).

Wide (persistent) splitting of S2 (S2 being split during both inspiration and expiration) is associated with right bundle branch block, pulmonary stenosis, pulmonary hypertension, and atrial septal defect.

A third commonly heard sound (designated sound S3 herein) coincides with rapid ventricular filling in early diastole. The sound S3 is sometimes referred to as ventricular gallop.

The sound S3 may be heard in healthy children and adolescents. It is considered abnormal when heard in patients over the age of 40, and is associated with conditions in which the ventricular contractile function is depressed, as occurs in congestive heart failure (CHF) and cardiomyopathy. The sound S3 also occurs in connection with conditions associated with volume overloading and dilation of the ventricles during diastole (mitral/tricuspid regurgitation or ventricular septal defect). The sound S3 also may sometimes be heard in the absence of heart disease, in connection with conditions associated with increased cardiac output, such as those noted above. A diagnosis known as pulsus alternans is characterized by a regular alternation of the force of the atrial pulse. Pulsus alternans almost always indicates the presence of severe left ventricular systolic dysfunction, and is usually associated with a gallop characteristic of S3.

A fourth part sound (designated herein as S4) can be heard that coincides with atrial contraction in late diastole. The sound S4 is sometimes referred to as atrial gallop.

The sound S4 is associated with conditions in which the ventricles lose their compliance and become stiff. The sound S4 may be heard during acute myocardial infarction. It is also commonly heard in connection with conditions associated with hypertrophy of the ventricles (e.g., systemic or pulmonary hypertension, aortic or pulmonary stenosis, and some cases of cardiomyopathy). It may also be heard in CHF.

Normal lung sounds occur in all parts of the chest area, including above the collarbones and at the bottom of the rib cage. Listening with a stethoscope (auscultation) may detect normal breathing sounds, decreased or absent breathing sounds, as well as abnormal breathing sounds.

Absent or decreased sounds reflect reduced airflow to a portion of the lungs, over-inflation of a portion of the lungs, air or fluid around the lungs, or increased thickness of the chest wall.

There are several types of abnormal breathing sounds, of which those known as rales, rhonchi, and wheezes are the most common. Rales (crackles or crepitations) are small clicking, bubbling or rattling sounds in the lung. These occur due to the opening and closing of the alveoli. Rales may further be described as moist, dry, fine and coarse. Ronchi are sounds that resemble snoring, and are produced when air movement through the large airways is obstructed or turbulent.

In the progression of CHF, it is possible to hear crackles when listening to the lung sounds.

Wheezes are high-pitched, musical sounds produced by narrowed airways, often occurring during expiration. Wheezes can be an indication, for example, of asthma.

It is also known to electronically analyze heart sounds to monitor the progression of diseases for optimizing or adjusting a pacing regimen. For example, U.S. Pat. No. 6,527,729 discloses monitoring the energy of the heart sound designated herein as S3, for monitoring the progression of CHF. A similar technique is disclosed in United States Patent Application Publication No. 2005/0149136. U.S. Pat. No. 6,792,308 analyzes ratios between the heart sounds designated herein as S1 and S2, and intervals therebetween to monitor cardiac status. Published PCT Application WO 01/56651 discloses a method for adjusting the A-V delay by monitoring the sounds designated herein as S1 and S2.

It is also known to electronically analyze lung sounds obtained from extracorporeally-placed microphones for the purpose of adjusting pulmonary therapy, as described in U.S. Pat. No. 6,116,241.

### SUMMARY OF THE INVENTION

An object of the present invention is to provide a cardiac assist device and method that make use of detection and analysis of heart and lung sounds for setting a cardiac therapy that is administered to the heart of a patient.

The above object is achieved in accordance with the present invention in a cardiac assist device and method wherein a microphone is placed in contact with the epicardium of the heart of a patient, and heart and lung sounds are simultaneously detected at the placement location of the microphone. The heart and lung sounds are automatically analyzed to set an appropriate cardiac therapy for the patient.

The microphone can be placed on the exterior of the epicardium, or can be placed inside the epicardium.

As used herein, the term “microphone” means any sensor that is capable of detecting vibrational frequencies of interest, including but not limited to audible frequencies.

As used herein, the phrase “setting a cardiac therapy” encompasses not only selection of a particular therapy, from among a number of available therapies, but also determining one or more parameters for the selected therapy.

The heart and lung sounds that are detected and analyzed can include, but are not limited to, the cardiac sounds S1, S2, S3 and S4 described above, as well as the lung sounds described above.

The cardiac therapy that is administered dependent on the simultaneous detection and subsequent analysis of the heart and lung sounds can be a pacing regimen and/or the delivery of antitachycardia pacing (ATP) and/or the delivery of one or more defibrillation pulses. ATP is typically administered to treat ventricular tachycardia (VT) that does not rise to the level of ventricular fibrillation (VF), which is treated with defibrillation pulses.

The heart and lung sounds are supplied in the form of an electrical signal from the microphone to appropriate evaluation circuitry in an implanted cardiac assist device. The signal itself and/or the analysis result obtained therefrom can be stored in a memory in the implanted device for subsequent readout by telemetry to an external device, such as an extracorporeal programmer for a more detailed review or analysis, as desired, by a cardiologist. Conventional electrical sensing of cardiac activity can be undertaken in parallel with the simultaneous detection of heart and lung sounds, using one or more electrodes that are implanted to interact with the heart. These sensed electrical signals can then be analyzed in a conventional manner to obtain a further analysis result, which can be used in combination with the analysis result of the simultaneous heart and lung sounds in order to set the therapy.

### DESCRIPTION OF THE DRAWINGS

FIG. 1 schematically illustrates a first embodiment of the inventive method and apparatus, with a microphone placed on the epicardium that communicates with a cardiac assist device implanted at an abdominal implantation site.

FIG. 2 schematically illustrates a first embodiment of the inventive method and apparatus, with a microphone placed inside the epicardium that communicates with a cardiac assist device implanted at an abdominal implantation site.

FIG. 3 schematically illustrates an embodiment of a cardiac assist device constructed and operating in accordance with the present invention.

FIG. 4 schematically illustrates a signal-processing flowchart as an embodiment for the operation of the cardiac assist device shown in FIG. 3.

### DESCRIPTION OF THE PREFERRED EMBODIMENTS

FIG. 1 schematically illustrates a first embodiment for placement of a microphone 1 relative to a heart 2 of a patient. In the embodiment shown in FIG. 1, the microphone 1 is placed on the epicardium of the heart, i.e., at an exterior placement location. The microphone 1 is connected via one or more leads to an implanted cardiac assist device, the housing 3 of which is illustrated in FIG. 1. In the embodiment of FIG. 1, the cardiac-assist device is shown implanted at an abdominal implantation site, but the cardiac-assist device could also be implanted at sub-clavian implantation site.

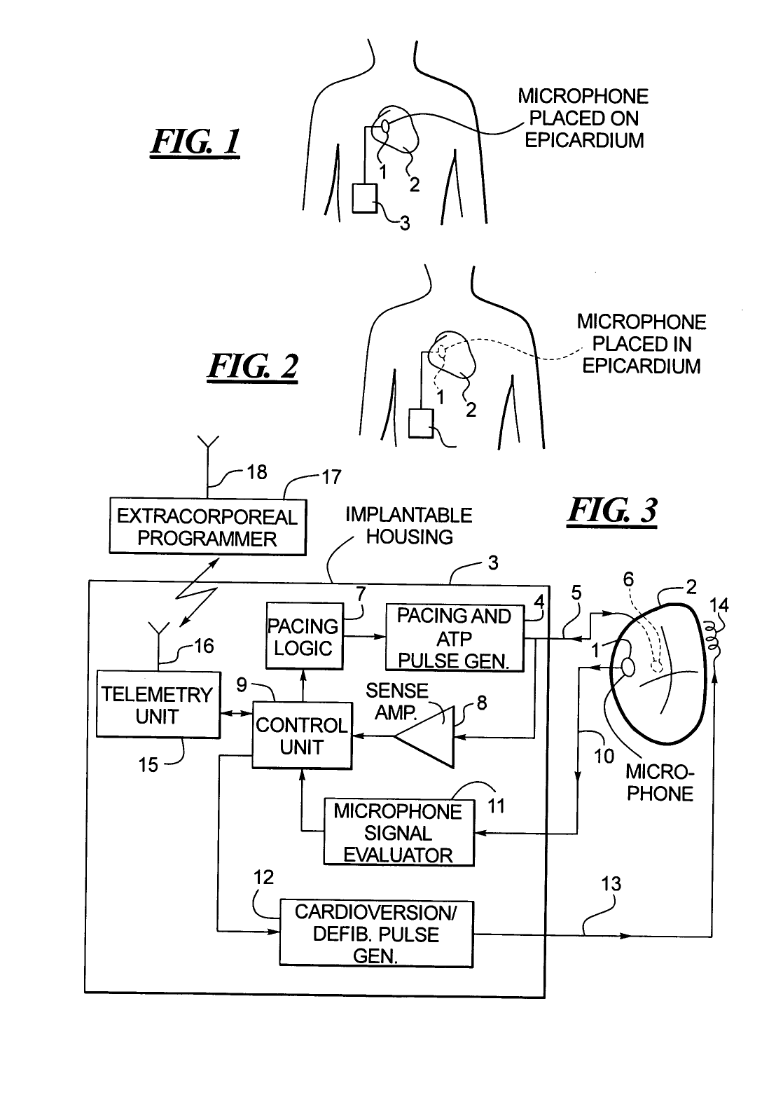

FIG. 2 illustrates a further embodiment of the method and device in accordance with the invention, wherein the microphone 1 is implanted at a placement site inside the epicardium.

FIG. 3 schematically illustrates the basic components of an embodiment of the cardiac-assist device according to the invention. The housing 3 contains a pulse generator 4 that generates pulses for pacing to treat bradycardia as well as pulses, as needed, for ATP. The pulse generator 4 is connected to a lead 5 that carries an electrode 6. Solely for exemplary purposes, a single electrode 6 is shown in the embodiment of FIG. 3, implanted in the right atrium. The invention, however, can be used for all types of known electrode configurations and implantation sites, including those for single chamber pacing, dual chamber pacing and biventricular pacing.

The pacing pulse generator 4 is operated by pacing logic 7.

The electrode 5 is also connected to a sense amplifier 8, which receives and detects signals via the lead 5 and the electrode 6 representing electrical activity of the heart 2. The output of the sense amplifier 8 is connected to a control unit 9, that provides control signals and setting to the pacing logic 7 for operating the pacing pulse generator 4.

In accordance with the invention, the microphone 1 communicates with a microphone signal evaluator 11 in the housing 3 via a lead 10. The placement site of the microphone 1 in the embodiment of FIG. 3 corresponds to the epicardial placement shown in FIG. 1, but all of the components shown in FIG. 3 can be used in the same manner in connection with the embodiment of FIG. 2, wherein the microphone 1 is placed inside the epicardium.

The microphone signal evaluator 11 evaluates an electrical signal generated by the microphone 1 that results from the simultaneous detection of heart and lung sounds at the placement site of the microphone 1. The microphone signal evaluator 11 makes use of the fact that blood is non-neutonian fluid that contains platelets in the form of red blood cells. Such a fluid is prone to create vortices as it flows through the circulatory system. A vortex is always accompanied by one or more pressure fluctuations. These fluctuations are picked up by the microphone 1. The frequency of the vortices is directly correlated to the flow velocity, and allows the microphone signal evaluator 11 to analyze the microphone signal to measure blood flow. As long as the simultaneously detected heart and lung sounds always originate from the same location, i.e., the placement site of the microphone 1, changes in blood flow can be determined.

It can be theorized that insufficient lubrication in the pericardial sac will cause the generation of friction-related sounds. These sounds can be expected to include short, high-frequency snaps from slipping movements. These sounds can also be detected by the microphone 1. The unique characteristic of this sound simplifies any filtering that may be necessary to extract such a sound from the overall microphone signal.

As noted above, the platelets (red blood cells) play an essential role in the generation of vortices. This means that the more red blood cells, the more vortices, and thus the stronger the microphone signal. Changes in signal strength are thus an indication of changes in hematocrit level. Many techniques for analyzing sounds (not necessarily devised for analyzing heart and lung sounds) are known, that involve time-domain analysis or frequency-domain analysis, or combinations thereof. Different heart rhythms create characteristic “footprints” depending on the origin and placement of the microphone 1. Based on these characteristics, discrimination among super-ventricular tachycardia (SVT) ventricular tachycardia (VT) and ventricular fibrillation (VF) can be made. Detection of beat-to-beat alternans during ischemia is another type of analysis that can be made.

It is also possible to detect atrial fibrillation (AF) by analyzing the simultaneously detected heart and lung sounds in the microphone signal evaluator 11. AF is a common condition, and although it is generally not life threatening by itself, it causes an increased risk of emboli, as well as discomfort, and weakens the ability of the heart to supply the body with oxygenated blood.

Moreover, AF may lead to several more serious conditions, and also is a predictor for several diseases. VF, unlike AF, is life threatening, and must be treated immediately, when detected. The sound of a fibrillating heart differs significantly from that of sinus rhythm, regardless of the heart rate, and thus offers a very useful complement or alternative to conventional electrical detection of fibrillation.

Another type of condition that can be detected by the analysis in the microphone signal evaluator 11 is the occurrence of post-ventricular contractions (PVC) and supra-ventricular contractions (SVC). When a PVC occurs, the filling is not complete, resulting in a quieter sounding valve than in the case of a normal beat. The following beat will then be more powerful than usual, and thus produce a louder sound, as there is an abnormal filling of the ventricle.

Moreover, irregular contractions of the heart that are not triggered by the sinus node or the normal conduction pathways of the heart often cause an extraordinary sound that differs from normal heartbeats. An example are so-called “cannon waves” that occur when the atrium contracts while the mitral valve is still closed, causing a backward rush of blood.

The control unit 9 can make use exclusively of the analysis or evaluation result from the microphone signal evaluator 11, but preferably also makes use of an analysis result of the electrical signal from the sense amplifier 8. The “final decision” for setting a cardiac-assist therapy that is made by the control unit 9 can be based on both of these analysis results, such as by a weighted combination. Alternatively, one analysis result can be used as a confirmation of the other analysis result.

The control unit appropriately controls the pacing logic 7 if and when the cardiac-assist therapy to be administered is a brady pacing regimen and/or ATP.

If the control unit 9 determines that a condition of VF exists, the control unit 9 then operates a cardioversion/defibrillation pulse generator 12 connected thereto that generates one or more defibrillation pulses, that are delivered to the heart 2 via a lead 13 connected to an electrode coil 14. As is known, the coil 14 is typically placed in the superior vena cava or the great vein.

It will be understood by those of ordinary skill in the field of designing cardiac assist devices that one or more suitable return paths must be provided for the electrode 6 and the electrode coil 14. Any suitable return electrode can be used, and therefore the return electrode or electrodes are not shown in FIG. 3.

Moreover, those of ordinary skill will also be aware that the housing 3 contains a battery (not shown) for supplying power to the components contained in the housing 3.

The control unit 9 is in communication with a telemetry unit 15 that has an antenna 16 allowing wireless communication with an extracorporeal programmer 17 that has an antenna 18. The control unit 9 can include, or be in communication with, a memory (not shown) in the implantable housing 3, so that the microphone signal, or the analysis results obtained therefrom, can be stored together with other data that are typically stored during the operation of a conventional cardiac-assist device. The stored data can be downloaded via the telemetry unit 15 at appropriate times to the extracorporeal programmer 17, so that the data can be evaluated in further detail, as needed, by a cardiologist. The data can be visually displayed at the extracorporeal programmer, and/or a printout of the data can be undertaken.

As described in the article “Presystolic Augmentation of Diastolic Heart Sounds in Atrial Fibrillation,” Bonner, Jr. et al., Am. J. Cardiol., Vol. 37, No. 3 (Mar. 4, 1976), pages 427-431, during atrial fibrillation the diastolic murmur of mitral stenosis can appear augmented during systole before the mitral valve closure sound. It is also known that during VF, no real contractions of the heart are occurring, and thus it is feasible to interpret a lack of “normal” heart sound, as usually occurs during sinus rhythm, as evidence of VF.

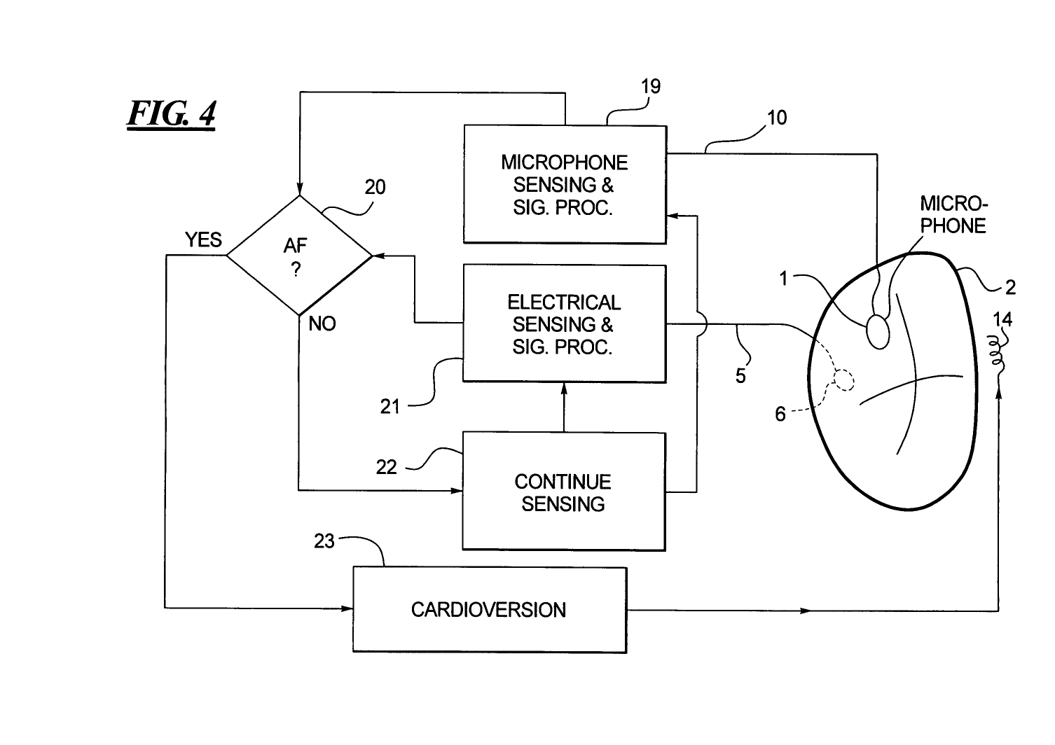

An example of analysis associated with AF that can be performed by the device of FIG. 3 is shown in FIG. 4. In this algorithm, microphone sensing and signal processing are represented by the block 19, and electrical sensing and signal processing are represented by the block 21. Respective analysis results from the blocks 19 and 21 are supplied to a decision stage 20, wherein it is determined whether AF exists. This can be accomplished, for example, by a template of healthy heart sound being recorded under normal conditions, and if a significant deviation from the template occurs, this is an indication of an arrhythmic event. If simultaneous indications from electrical sensors and an activity sensor also are present, it is very likely that an arrhythmia has begun.

If no occurrence of AF is determined to exist, sensing continues as before, as indicated by the block 22. If AF is determined to be present, and is serious enough to require cardiac-assist therapy, one or more cardioversion pulses can be administered, as indicated by the block 23.

Although modifications and changes may be suggested by those skilled in the art, it is the intention of the inventors to embody within the patent warranted hereon all changes and modifications as reasonably and properly come within the scope of their contribution to the art.

We claim as our invention:

1. A cardiac-assist device comprising:
an implantable housing;
a microphone, adapted for placement at a placement location in contact with the epicardium of a heart, that detects heart and lung sounds simultaneously from said placement location, said simultaneous heart and lung sounds being represented in an electrical signal from the microphone;
cardiac therapy circuitry in said housing that generates a cardiac therapy;
an electrode arrangement connected to the cardiac therapy circuitry and adapted to interact with the heart to apply said cardiac therapy thereto; and
evaluation and control circuitry that automatically evaluates said signal from said microphone and that controls said cardiac therapy circuitry to set said cardiac therapy dependent on the simultaneous heart and lung sounds represented in said signal.

2. A cardiac assist device as claimed in claim 1 wherein said cardiac therapy circuitry comprises a pacing pulse generator.

3. A cardiac assist device as claimed in claim 1 wherein said cardiac therapy circuitry comprises a cardioversion/defibrillation pulse generator.

4. A cardiac assist device as claimed in claim 1 comprising electronic sensing circuitry, connected to said electrode arrangement that senses electrical activity of the heart, and wherein said evaluation and control circuitry sets said cardiac therapy additionally dependent on the electrical activity sensed by said electrical sensing circuitry.

5. A method for providing cardiac therapy to a patient, comprising the steps of:
implanting a microphone at a placement location in contact with the epicardium of the heart of the patient;
detecting heart and lung sounds in the patient simultaneously from said placement location with said microphone and generating an electronic microphone signal representing said simultaneous heart and lung sounds;
electronically analyzing said simultaneous heart and lung sounds in said microphone signal to obtain an analysis result; and
automatically setting a cardiac therapy dependent on said analysis result, and administering said cardiac therapy to the patient.

6. A method as claimed in claim 5 comprising administering pacing pulses to the patient as said therapy.

7. A method as claimed in claim 5 comprising administering pulses selected from the group consisting of cardioversion pulses and defibrillation pulses as said therapy.

8. A method as claimed in claim 5 comprising detecting electrical activity of the heart of the patient and generating a further analysis result dependent on the detected electrical activity, and setting said cardiac therapy dependent on both said analysis result and said further analysis result.

9. A method as claimed in claim 5 wherein the step of electronically analyzing said simultaneous heart and lung sounds comprises analyzing a loudness of said simultaneous heart and lung sounds.

10. A method as claimed in claim 5 wherein the step of electronically analyzing said simultaneous heart and lung sounds comprises analyzing a repetitive characteristic of said simultaneous heart and lung sounds.

11. A method as claimed in claim 10 wherein said repetitive characteristic is a beat-to-beat characteristic of the heart.

12. A method as claimed in claim 5 wherein the step of electronically analyzing said simultaneous heart and lung sounds comprises analyzing variations in loudness of said simultaneous heart and lung sounds.

13. A method as claimed in claim 5 wherein the step of electronically analyzing said simultaneous heart and lung sounds comprises analyzing said simultaneous heart and lung sounds to detect rales.

14. A method as claimed in claim 5 wherein the step of electronically analyzing said simultaneous heart and lung sounds comprises analyzing said simultaneous heart and lung sounds to detect rhonchi.

15. A method as claimed in claim 5 wherein the step of electronically analyzing said simultaneous heart and lung sounds comprises analyzing said simultaneous heart and lung sounds to detect wheezes.

16. A method as claimed in claim 5 wherein the step of electronically analyzing said simultaneous heart and lung sounds comprises analyzing said simultaneous heart and lung sounds to detect atrial fibrillation.

17. A method as claimed in claim 5 wherein the step of electronically analyzing said simultaneous heart and lung sounds comprises analyzing said simultaneous heart and lung sounds to detect ventricular fibrillation.

18. A method as claimed in claim 5 wherein the step of electronically analyzing said simultaneous heart and lung sounds comprises analyzing said simultaneous heart and lung sounds to detect ventricular tachycardia.

19. A method as claimed in claim 5 wherein the step of electronically analyzing said simultaneous heart and lung sounds comprises analyzing said simultaneous heart and lung sounds to detect post-ventricular contractions.

20. A method as claimed in claim 5 wherein the step of electronically analyzing said simultaneous heart and lung sounds comprises analyzing said simultaneous heart and lung sounds to detect supra-ventricular contractions.

21. A method as claimed in claim 5 wherein the step of electronically analyzing said simultaneous heart and lung sounds comprises analyzing said simultaneous heart and lung sounds to detect cannon waves.

22. A method as claimed in claim 5 wherein the step of electronically analyzing said simultaneous heart and lung sounds comprises analyzing said simultaneous heart and lung sounds to discriminate from among supra-ventricular tachycardia, ventricular tachycardia and ventricular fibrillation.

23. A method as claimed in claim 5 wherein the step of electronically analyzing said simultaneous heart and lung sounds comprises analyzing said simultaneous heart and lung sounds to detect ventricular gallop.

24. A method as claimed in claim 5 wherein the step of electronically analyzing said simultaneous heart and lung sounds comprises analyzing said simultaneous heart and lung sounds to detect atrial gallop.

25. A method as claimed in claim 5 comprising placing said microphone at a placement location on the epicardium.

26. A method as claimed in claim 5 comprising placing said microphone at a placement location inside the epicardium.
